(19) United States
(12) Patent Application Publication
(10) Pub. No.: US 2010/0144835 A1
(43) Pub. Date: Jun. 10, 2010

(54) INHIBITION OF GPR4

(75) Inventors: Klaus Seuwen, Basel (CH); Eric Billy, Basel (CH); Thomas Suply, Basel (CH); Lorenza Wyder, Basel (CH); Janet Dawson King, Basel (CH); Marie-Gabrielle Ludwig, Basel (CH); Matthias Mueller, Basel (CH); Puneeta Nath, Horsham (GB); Carol Elizabeth Jones, Horsham (GB)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG

(21) Appl. No.: 12/518,709

(22) PCT Filed: Dec. 13, 2007
(86) PCT No.: PCT/EP07/63899

§ 371 Date: Jun. 11, 2009

(30) Foreign Application Priority Data
Dec. 15, 2006 (EP) 06126205.1

(51) Int. Cl. A61K 31/7052; C07H 21/02; A01K 67/033; A61P 9/00; C12Q 1/02

(52) U.S. Cl. 514/44 A; 536/24.5; 800/13; 800/9; 800/10; 800/11; 435/29

(57) ABSTRACT

The present invention relates to the use of a GPR4 inhibitor for the manufacture of a medicament for the inhibition of angiogenesis, for instance for the inhibition of tumour growth in the treatment of cancer. In a preferred embodiment, said inhibitor is a siRNA, preferably double-stranded.

In addition, the present invention further encompasses non-human animals wherein the GPR4 has been inactivated, for instance a knock-out mouse lacking GPR4, and the use of said animals as an experimental model for angiogenesis and for screening for compounds modulating angiogenesis.

### 1. FIELD OF THE INVENTION

The present invention relates to the use of a GPR4 inhibitor for the manufacture of a medicament for the inhibition of angiogenesis, for instance for the inhibition of tumour growth in the treatment of cancer or for the treatment of arthritis. In a preferred embodiment, said inhibitor is a siRNA.

The present invention also relates to the non-human animals wherein the GPR4 has been inactivated and the use of said animals as an experimental model for angiogenesis and for screening for compounds modulating angiogenesis.

### 2. BACKGROUND OF THE INVENTION

GPR4 belongs to a protein family comprising 3 closely related G protein-coupled receptors (GPCRs): GPR4, OGR1/GPR68 and TDAG8/GPR65. We have previously shown that OGR1 as well as GPR4 sense extracellular protons and stimulate intracellular second messengers upon exposure to slightly acidic pH¹. Similarly, TDAG8 has been identified as a proton-sensing receptor^{2,3}. Half maximal activation of these receptors is observed in the physiological range, around pH 7.4, and highest activity is observed at pH 6.8. In gene expression profiling studies, we found a strong correlation between the expression of GPR4 mRNA and marker genes for endothelial cells. Earlier reports had already described an important role for GPR4 in endothelial cell function, but these findings were seen in relation to the presumed ligand sphingosylphosphorylcholine (SPC)^{4,5}.

Angiogenesis, the formation of new blood vessels, is a hallmark of cancer, allowing tumours to grow beyond 1-3 mm³ in size and facilitating local invasion and metastasis. It is induced by aberrant expression of angiogenic growth factors such as VEGF (vascular endothelial growth factor) but also by local alteration of the tumour microenvironment through hypoxia, glucose deprivation, and oxidative and mechanical stress⁶. Recently, the first anti-VEGF therapy was approved for the treatment of colorectal cancer, validating the notion that angiogenesis is an important target for cancer therapy.

Tumours might have an acidic pH compared to normal tissues⁷. As a response to hypoxia tumour cells increase their glycolytic rate to produce energy and thereby acidify the extracellular space^{8,9}. Most tumours upregulate glycolysis as can be observed by FdG (fluorodesoxyglucose) PET (positron-emission tomography), a commonly used imaging method to diagnose tumours¹⁰. By adapting to hypoxia and acidosis, tumour cells survive under conditions that are not tolerated by normal cells and this feature may correlate with invasive potential. Normal cells undergo necrosis or apoptosis after prolonged exposure to an acidic microenvironment¹¹.

Little is known about how cells can adapt to extracellular acidosis, and the interplay between hypoxia and acidosis in the regulation of angiogenesis is not yet fully understood¹².

### 3. SUMMARY OF THE INVENTION

The present inventors have analyzed expression of GPR4 on endothelial cells and show pH-dependent cAMP formation in these cells. The present inventors demonstrate that in HUVECs (human umbilical vein endothelial cells) the cAMP response is abrogated by GPR4-specific siRNAs, indicating that GPR4 is responsible for pH-sensing. These findings indicate a promising new approach for controlling angiogenesis.

To gain a better understanding of the role of GPR4 in physiology, GPR4-deficient mice were generated by the present inventors. Surprisingly these animals are viable and fertile and do not show major abnormalities, indicating that GPR4 is not critical during development. However, GPR4-deficient mice show significantly reduced responses to VEGF-driven but not to bFGF-driven angiogenesis when subjected to a growth factor implant angiogenesis model. In addition, tumour growth is reduced in GPR4-deficient mice compared to wild-type mice in two different orthotopic tumour models. Reduced tumour growth correlates with impaired vessel structure as well as reduced VEGFR2 levels in GPR4-deficient mice. Without wishing to be bound by theory, the present inventors therefore conclude that acidosis is sensed by endothelial cells via GPR4, and that this signal can modulate pathological angiogenesis. These findings indicate a promising new approach for controlling angiogenesis.

The present invention hence relates to the use of a GPR4 inhibitor for the manufacture of a medicament for the inhibition of angiogenesis, for instance for the inhibition of tumour-growth in the treatment of cancer, macular degeneration, psoriasis, arthritis, multiple sclerosis or atherosclerosis. In a preferred embodiment, said inhibitor is a siRNA, preferably double-stranded.

Particularly preferred are double-stranded siRNA molecules targeted against human GPR4, said double-stranded siRNA molecules having the following sequences:

| | |
|---|---|
| sense: | |
| 5′-GCGCTGTGTCCTATCTCAAdTdT-3′ | (SEQ ID NO: 1) |
| and | |
| anti-sense: | |
| 5′-TTGAGATAGGACACAGCGCdAdG-3′, | (SEQ ID NO: 2) |
| or | |
| sense: | |
| 5′-CCATGTCTGGCCAGATAAAdTdT-3′ | (SEQ ID NO: 4) |
| and | |
| anti-sense: | |
| 5′-TTTATCTGGCCAGACATGGdCdG-3′, | (SEQ ID NO: 5) |
| or | |
| sense: | |
| 5′-CATAAGACCGCAATTCTAAdTdT-3′ | (SEQ ID NO: 7) |
| and | |
| anti-sense: | |
| 5′-TTAGAATTGCGGTCTTATGdTdT-3′. | (SEQ ID NO: 8) |

The present invention also encompasses the treatment of patients with the GPR4 inhibitors of the invention.

The instant invention moreover also encompasses siRNA molecules comprising the sequence of SEQ ID NO:1-20, which siRNA are suitable for use in e.g. human and/or mice.

In addition, the present invention further encompasses a knock-out non-human animal lacking GPR4 and the use of said non-human animal as an experimental model for angiogenesis, cancer or arthritis, and for screening for compounds modulating angiogenesis, cancer or arthritis.

### 4. BRIEF DESCRIPTION OF THE FIGURES

FIG. 1: GPR4 is highly expressed in endothelial cells

a) GPR4 expression in various types of endothelial cells (dark grey), normal cells (light grey) and tumour cell lines (white) was determined by microarray experiments. The MAS5 normalized values of GPR4 expression levels are shown (n=2-3 per sample). HUVECs were from Vectec (VT) or Promocell (PC); HPAEC: primary human pulmonary aortic endothelial cells; HMVEC: primary human microvascular endothelial cells, DU145 human prostate cancer cells; HeLa human cervical cancer cells b) Expression of GPR4 was confirmed by RT-PCR in several human and mouse endothelial cells, but was absent in the tested tumour cells. GAPDH was used as an internal control. MS1: mouse pancreatic endothelial cell line, 4T1: mouse breast tumour cell line, CT26: mouse colon tumour cell line.

FIG. 2: GPR4 expressing cells respond to extracellular acidification by cAMP production, a reaction which can be blocked by GPR4-specific siRNAs.

The production of cAMP in response to extracellular pH changes was assessed a) in HeLa cells stably transfected with GPR4. b-d) in HUVECS; in b forskolin (FSK, an adenylyl cyclase agonist) was used to increase the assay window. d) GPR4-specific, but not control siRNAs inhibit the pH-dependent cAMP production (n=2-3 measurements per point). IBMX (a phophodiesterase inhibitor) was used to stabilize cAMP.

FIG. 3: Generation of GPR4-deficient mice

a) Targeting construct and strategy used to generate GPR4-deficient mice by homologous recombination in ES-cells. P denote the primers used to generate the constructs (see Materials and Methods). b) Southernblot on SacI-digested genomic DNA from a wild type and a GPR4-deficient mouse with probe depicted in panel a. c) RT-PCR for GPR4 in different organs of wild type and GPR4-deficient mice. Clathrin-2K (Clathk) was used as control. d) RT-PCR for GPR4 in primary lung endothelial cells (Lung ECs) isolated from wild type or GPR4-deficient mice, GAPDH was used as control.

FIG. 4: GPR4 deficiency results in impaired response to VEGF-driven angiogenesis

a) Teflon chambers containing agar with or without growth factor were implanted on the back of wild type or GPR4-deficient female mice. After 4 days the implant was removed and the tissue which formed around the chamber was weighed. b) The endothelial cell specific marker Tie2 was measured by ELISA as a way to quantify vascularity. c) Physical appearance of the different implants. d) Teflon chambers containing agar with or without growth factor together with siRNAs were implanted on the back of wild type female mice. After 3 days the implant was removed and the tissue which formed around the chamber was weighed e) The amount of Tie2 marker was measured by ELISA in the chambers containing growth factor and siRNAs (n=6 per group, *P≦0.05 WT versus GPR4-KO; **P≦0.05 compared to PBS).

FIG. 5: GPR4-KO mice show reduced tumour growth in two different orthotopic tumour models

a) Syngeneic 4T1 breast tumour cells were implanted orthotopically into the fat pad of wild type and GPR4-deficient mice. Tumour growth was measured over time with calipers b) Mice were sacrificed after 21 days and 4T1 tumour weight was measured (n=6 mice per group, *P≦0.05 WT versus GPR4-KO). c) Syngeneic CT26 colon tumour cells were implanted orthotopically into the caecum of WT and GPR4-deficient mice. Animals were sacrificed after 20 days and tumour weight was measured. d) Physical appearance of the colon tumours. (n=8-10 per group, *P≦0.05 WT versus GPR4-KO).

FIG. 6:

a) Examples of the fragile and disrupted appearance of blood vessels in CT26 tumours grown in GPR4-deficient mice as compared to wild type mice (CD31 staining, bar 32 50 μm). b) Vessel density was assessed by counting CD31 positive vessels on the whole area of tumours grown in WT or GPR4-deficient mice (n=4-5 per group). C) Vessel length. d) Percentage of proliferating, Ki67-positive cells in tumours grown in wild type versus GPR4-deficient mice (n=5 per group; *P<0.001).

FIG. 7: GPR4 deficiency results in downregulation of VEGFR2 but not of other endothelial cell markers

a-b) VEGFR2 8a) and Tie2 (b) expression was measured in lungs of WT or GPR4-deficient mice by ELISA. c) EphB4 and VE-Cadherin expression was measured in lungs of WT and GPR4-deficient mice by Western blot. Tubulin was used as control for equal protein loading. (n=6 per group, *P<0.05). d) HUVEC cells were transfected with different siRNAs and surface expression of VEGFR2 was assessed by FACS analysis 48h after transfection.

FIG. 8: GPR4-deficient mice have a marked and significant inhibition of knee swelling as compared to wild type mice

FIG. 9: Mean numbers of total cells, macrophages, lymphocytes and neutrophils in bronchoalveolar lavage fluid following acute cigarette smoke exposure in GPR4−/− and Balb/C mice (n=7/8). *p<0.05, **p<0.01, ***p<0.0001. [symbol] =Balb/C Sham-exposed mice, [symbol] =GPR4−/− Sham-exposed mice, [symbol] ==Balb/C Smoke-exposed mice [symbol] =GPR4−/− Smoke-exposed mice.

FIG. 10. Mean numbers of total cells, macrophages, lymphocytes and neutrophils in bronchoalveolar lavage fluid following sub-chronic cigarette smoke exposure in GPR4−/− and Balb/C mice (n=7/8). *p<0.05, **p<0.01, ***p<0.0001. Data shown as mean±SEM. [symbol] =Balb/C Sham-exposed mice, [symbol] =GPR4−/− Sham-exposed mice, [symbol] =Balb/C Smoke-exposed mice, [symbol] =GPR4−/− Smoke-exposed mice.

FIG. 11: MIP-2 levels in lung tissue, following sub-chronic cigarette smoke exposure in GPR4−/− and Balb/C mice (n=7/8). *p<0.05, **p<0.01, ***p<0.0001. Data shown as mean±SEM. [symbol] =Balb/C Sham-exposed mice, [symbol] =GPR4−/− Sham-exposed mice, [symbol] =Balb/C Smoke-exposed mice, [symbol] =GPR4−/− Smoke-exposed mice.

FIG. 12: Airway hyperresponsiveness in GPR4−/− and Balb/C mice (n=7/8) following ovalbumin sensitization and ovalbumin exposure. PC₃₀₀ was calculated by the interpolation of the log concentration-lung resistance curve from individual animals. *p<0.05, **p<0.01, ***p<0.0001. Data shown as mean±SEM.

FIG. 13: Mean numbers of total cells, macrophages, lymphocytes and neutrophils in bronchoalveolar lavage fluid following chronic cigarette smoke exposure in GPR4−/− and Balb/C mice (n=7/8). *p<0.05, **p<0.01, ***p<0.0001. Data shown as mean±SEM. [symbol] =Balb/C PBS-exposed mice, [symbol] =GPR4 PBS-exposed mice, [symbol] =Balb/C Ovalbumin-exposed mice, [symbol] =GPR4−/− Ovalbumin-exposed mice.

### 5. DETAILED DESCRIPTION OF THE INVENTION

The G protein-coupled receptor GPR4 is activated by acidic pH, but little is known regarding its physiological role. The present inventors observed a surprisingly high correlation of GPR4 mRNA expression with endothelial marker genes, and demonstrate expression and function of GPR4 in primary human vascular endothelial cells. The present invention hence relates to the use of a GPR4 inhibitor for the treatment of a subject or for the manufacture of a medicament for the inhibition of angiogenesis, for instance for the inhibition of tumour-growth in the treatment of cancer, macular degeneration, psoriasis, arthritis, multiple sclerosis or atherosclerosis. In a preferred embodiment, said inhibitor is a siRNA. Also part of the invention are GPR4-deficient mice, which surprisingly are viable and fertile. These animals show a significantly reduced angiogenic response to VEGF, but not to bFGF in a growth factor implant model. In addition, growth of injected tumour cells is markedly reduced in mice lacking GPR4. Histological analysis of tumours indicated reduced tumour cell proliferation, reduced vessel density, and altered vessel morphology. Moreover, GPR4 deficiency results in reduced VEGFR2 levels in endothelial cells, accounting, at least in part, for the observed phenotype. These data show that endothelial cells sense local tissue acidosis via GPR4 and that this signal is required to generate a full angiogenic response to VEGF, thus providing a target for the therapy of e.g. tumours, macular degeneration, psoriasis, arthritis, multiple sclerosis or atherosclerosis. In addition, the present inventors have discovered that GPR4-deficient animals can be advantageously used as an experimental model for arthritis.

RNAi is the process of sequence specific post-transcriptional gene silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted knockdown′ of gene expression.

“siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

Preferably, the siRNA molecule is substantially identical with at least a region of the coding sequence of the GPR4 to enable down-regulation of the gene. Preferably, the degree of identity between the sequence of the siRNA molecule and the targeted region of the GPR4 gene is at least 60% sequence identity, preferably, at least 75% sequence identity, preferably at least 85% identity; preferably at least 90% identity; preferably at least 95% identity; preferably at least 97% identity; and most preferably, at least 99% identity.

Calculation of percentage identities between different amino acid/polypeptide/nucleic acid sequences may be carried out as follows. A multiple alignment is first generated by the ClustaIX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)* 100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesized de novo, or may be native amino acid/polypeptide/nucleic acid sequence, or a derivative thereof A substantially similar nucleotide sequence will be encoded by a sequence which hybridizes to any of the nucleic acid sequences referred to herein or their complements under stringent conditions. By stringent conditions, we mean the nucleotide hybridizes to filter-bound DNA or RNA in 6×sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid.

The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA.

Frequently, automatically generated alignments require manual alignment, exploiting the trained user's knowledge of the protein family being studied, e.g., biological knowledge of key conserved sites. One such alignment editor programs is Align (http://www.gwdg. de/dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable.

Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs. washington.edu/phylip.html) using the “Similarity Table” option as the model for amino acid substitution (P). For DNA/RNA, an identical option exists within the DNADIST program of PHYL1P.

The dsRNA molecules in accordance with the present invention comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer. In a preferred embodiment, the inhibitor is a siRNA molecule and comprises between approximately 5 bp and 50 bp, more preferably between 10 by and 35 bp, even more preferably between 15 by and 30 bp, and yet still more preferably, between 18 by and 25bp. Most preferably, the siRNA molecule comprises more than 20 and less than 23 bp.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention display a high in vivo stability and may be particularly suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918. Suitable modifications for delivery include chemical modifications can be selected from among:
a) a 3′ cap;
b) a 5′ cap,
c) a modified internucleoside linkage; or
d) a modified sugar or base moiety.

Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides) with for instance phosphorothioates, chiral phosphorothioates or phosphorodithioates.

End modifications sometimes referred to herein as 3′ caps or 5′ caps may be of significance. Caps may consist of simply adding additional nucleotides, such as “T-T” which has been found to confer stability on an siRNA. Caps may consist of more complex chemistries which are known to those skilled in the art.

Design of a suitable siRNA molecule is a complicated process, and involves very carefully analyzing the sequence of the target mRNA molecule. On exemplary method for the design of siRNA is illustrated in WO2005/059132. Then, using considerable inventive endeavour, the inventors have to choose a defined sequence of siRNA which has a certain composition of nucleotide bases, which would have the required affinity and also stability to cause the RNA interference.

Preferred siRNAs of the invention are:

| | |
|---|---|
| sense: | |
| 5′-GCGCTGTGTCCTATCTCAAdTdT-3′ | (SEQ ID NO: 1) |
| anti-sense: | |
| 5′-TTGAGATAGGACACAGCGCdAdG-3′ | (SEQ ID NO: 2) |
| target (human): | |
| GCGCTGTGTCCTATCTCAA | (SEQ ID NO: 3) |
| sense: | |
| 5′-CCATGTCTGGCCAGATAAAdTdT-3′ | (SEQ ID NO: 4) |
| anti-sense: | |
| 5′-TTTATCTGGCCAGACATGGdCdG-3′ | (SEQ ID NO: 5) |
| target (human): | |
| CCATGTCTGGCCAGATAAA | (SEQ ID NO: 6) |
| sense: | |
| 5′-CATAAGACCGCAATTCTAAdTdT-3′ | (SEQ ID NO: 7) |
| anti-sense: | |
| 5′-TTAGAATTGCGGTCTTATGdTdT-3′ | (SEQ ID NO: 8) |
| target (human): | |
| CATAAGACCGCAATTCTAA | (SEQ ID NO: 9) |
| sense: | |
| 5′-GGAGGTAGGACTAACAATAdTdT-3′ | (SEQ ID NO: 10) |
| anti-sense: | |
| 5′-TATTGTTAGTCCTACCTCCdCdT-3′ | (SEQ ID NO: 11) |
| target (mouse): | |
| GGAGGTAGGACTAACAATA | (SEQ ID NO: 12) |
| sense: | |
| 5′-GGGTCTGAAGGGGGAACAAdTdT-3′ | (SEQ ID NO: 13) |
| anti-sense: | |
| 5′-TTGTTCCCCCTTCAGACCCdTdG-3′ | (SEQ ID NO: 14) |
| target (mouse): | |
| GGGTCTGAAGGGGGAACAA. | (SEQ ID NO: 15) |

Also encompassed are siRNA molecules comprising the sequences:

| | |
|---|---|
| TTGAGATAGGACACAGCGC | (SEQ ID NO: 16) |
| TTTATCTGGCCAGACATGG | (SEQ ID NO: 17) |
| TTAGAATTGCGGTCTTATG | (SEQ ID NO: 18) |
| TATTGTTAGTCCTACCTCC | (SEQ ID NO: 19) |
| TTGTTCCCCCTTCAGACCC | (SEQ ID NO: 20) |

The siRNA molecule may be either synthesized de novo, or produced by a micro-organism. For example, the siRNA molecule may be produced by bacteria, for example, E. coli. Methods for the synthesis of siRNA, including siRNA containing at least one modified or non-natural ribonucleotides are well known and readily available to those of skill in the art. For example, a variety of synthetic chemistries are set out in published PCT patent applications WO2005021749 and WO200370918, both incorporated herein by reference. The reaction may be carried out in solution or, preferably, on solid phase or by using polymer supported reagents, followed by combining the synthesized RNA strands under conditions, wherein a siRNA molecule is formed, which is capable of mediating RNAi.

It should be appreciated that siNAs (small interfering nucleic acids) may comprise uracil (siRNA) or thyrimidine (siDNA). Accordingly the nucleotides U and T, as referred to above, may be interchanged. However it is preferred that siRNA is used.

The inventors tested each of these siRNA molecules by the methods as described in the Examples and demonstrated that these inhibitors were effective for reducing GPR4 expression, reducing angiogenesis and that these siRNA molecules of the invention are thereby effective for treating cancer, in particular for the inhibition of tumour growth.

Gene-silencing molecules, i.e. inhibitors, used according to the invention are preferably nucleic acids (e.g. siRNA or antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators).

The gene-silencing molecule may be either synthesized de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene silencing in the target cell.

The molecule may be produced by a vector harbouring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage, or virus DNA. In addition to, or instead of using the vector to synthesize the gene-silencing molecule, the vector may be used as a delivery system for transforming a target cell with the gene silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favour targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, endothelial cells. The promoter may be constitutive or inducible.

Alternatively, the gene silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentivirus and the like).

Alternatively a “naked” siRNA or antisense molecule may be inserted into a subject's cells by a suitable means e.g. direct endocytotic uptake.

The gene silencing molecule may also be transferred to the cells of a subject to be treated by either transfection, infection, microinjection, cell fusion, protoplast fusion or ballistic bombardment. For example, transfer may be by: ballistic transfection with coated gold particles; liposomes containing an siNA molecule; viral vectors comprising a gene silencing sequence or means of providing direct nucleic acid uptake (e.g. endocytosis) by application of the gene silencing molecule directly.

In a preferred embodiment of the present invention siNA molecules may be delivered to a target cell (whether in a vector or “naked”) and may then rely upon the host cell to be replicated and thereby reach therapeutically effective levels. When this is the case the siNA is preferably incorporated in an expression cassette that will enable the siNA to be transcribed in the cell and then interfere with translation (by inducing destruction of the endogenous mRNA coding GPR4).

Inhibitors according to any embodiment of the present invention may be used in a monotherapy (e.g. use of siRNAs alone). However it will be appreciated that the inhibitors may be used as an adjunct, or in combination with other, e.g., cancer therapies (e.g. radiotherapy, conventional chemotherapy or even in conjunction with other oncogene gene silencing strategies). For instance, a combination therapy may comprise a gene silencing molecule according to the invention and a course of radiotherapy.

The inhibitors according to the invention may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal suffering from e.g. cancer or at risk of developing a cancer. It will be appreciated that the vehicle of the composition of the invention should be one which is well tolerated by the subject to whom it is given, and preferably enables delivery of the inhibitor to the target site.

The inhibitors according to the invention may be used in a number of ways.

For instance, systemic administration may be required in which case the compound may be contained within a composition that may, for example, be administered by injection into the blood stream. Injections may be intravenous (bolus or infusion), subcutaneous, intramuscular or a direct injection into the target tissue (e.g. an intraventricular injection—when used in the brain). The inhibitors may also be administered by inhalation (e.g. intranasally) or even orally (if appropriate).

The inhibitors of the invention may also be incorporated within a slow or delayed release device. Such devices may, for example, be inserted at the site of a tumour, and the molecule may be released over weeks or months. Such devices may be particularly advantageous when long term treatment with an inhibitor according to the invention is required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, the mode of administration, and the advancement or severity of the cancer.

Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue.

Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 pg/kg of body weight and 0.5 g/kg of body weight of an inhibitor according to the invention may be used for the treatment of cancers, depending upon which specific inhibitor is used. When the inhibitor is an siRNA molecule, the daily dose may be between 1 pg/kg of body weight and 100 mg/kg of body weight, and more preferably, between approximately 10 pg/kg and 10 mg/kg, and even more preferably, between about 50 pg/kg and 1 mg/kg.

When the inhibitor (e.g. siNA) is delivered to a cell, daily doses may be given as a single administration (e.g. a single daily injection).

Alternatively, some inhibitors, or cancer conditions, may require administration twice or more times during a day. As an example, siNA's according to the invention may be administered as two (or more depending upon the severity of the condition) daily doses of between 0.1 mg/kg and 10 mg/kg (i.e. assuming a body weight of 70 kg). A patient receiving treatment may take a first dose upon waking and then a second dose in the evening (if on a two dose regime) or at 3 or 4 hourly intervals thereafter. Alternatively, a slow release device may be used to provide optimal doses to a patient without the need to administer repeated doses.

Medicaments according to the invention should comprise a therapeutically effective amount of an inhibitor of GPR4 and a pharmaceutically acceptable vehicle.

The following definitions are used throughout this specification and the claims.

An “isolated nucleic acid sequence” means that the material is removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated, even if subsequently reintroduced into the natural system. Such polynucleotides could be part of a vector and/or such polynucleotides could be part of a composition, and still be isolated in that such vector or composition is not part of its natural environment.

A “nucleic acid vector” is a nucleic acid sequence designed to be propagated and or transcribed upon exposure to a cellular environment, such as a cell lysate or a whole cell. A “gene therapy vector” refers to a nucleic acid vector that also carries functional aspects for transfection into whole cells, with the intent of increasing expression of one or more genes and/or proteins. In each case such vectors usually contain a “vector propagation sequence” which is commonly an origin of replication recognized by the cell to permit the propagation of the vector inside the cell. A wide range of nucleic acid vectors and gene therapy vectors are familiar to those skilled in the art.

As used herein, the phrases “therapeutically effective amount” and “prophylactically effective amount” refer to an amount that provides a therapeutic benefit in the treatment, prevention, or management of pathological processes. The specific amount that is therapeutically effective can be readily determined by ordinary medical practitioner, and may vary depending on factors known in the art, such as, e.g. the type of pathological processes, the patient's history and age, the stage of pathological processes, and the administration of other agents in combination. For example, a “therapeutically effective amount” is any amount of an inhibitor according to the invention which, when administered to a subject inhibits cancer growth.

As used herein, a “pharmaceutical composition” comprises a pharmacologically effective amount of a therapeutic agent of the invention and a pharmaceutically acceptable carrier. As used herein, “pharmacologically effective amount,” “therapeutically effective amount” or simply “effective amount” refers to that amount of an agent effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “transformed cell” is a cell into which a vector has been introduced from which a dsRNA molecule may be expressed. A cell comprising a nucleic acid which is supplied exogenously, such as the agents of this invention, whether transfected transiently or stably, is also considered a transformed cell.

In practicing the present invention, many conventional techniques in molecular biology, microbiology, and recombinant DNA are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Cabs eds., Cold Spring Harbor Laboratory); and Methods in Enzymology Vol. 154 and Vol. 155 (Wu and Grossman, and Wu, eds., respectively).

A “subject” may be a vertebrate, mammal, domestic animal or human being. It is preferred that the subject to be treated is human. When this is the case the inhibitors may be designed such that they are most suited for human therapy. However it will also be appreciated that the inhibitors may also be used to treat other animals of veterinary interest (e.g. horses, dogs or cats). Alternatively, the subject might be a mouse, for instance in an experimental model. Furthermore, in an another experimental model said subject might be a single cell or a population of cultured cells.

A “pharmaceutically acceptable vehicle” as referred to herein is any physiological vehicle known to those of ordinary skill in the art useful in formulating pharmaceutical compositions.

Preferably, the medicament comprises approximately 0.1% (w/w) to 90% (w/w) of the inhibitor, and more preferably, 1% (w/w) to 10% (w/w). The rest of the composition may comprise the vehicle.

In a preferred embodiment, the pharmaceutical vehicle is a liquid and the pharmaceutical composition is in the form of a solution. In another embodiment, the pharmaceutical vehicle is a gel and the composition is in the form of a cream or the like.

Liquid pharmaceutical compositions which are sterile solutions or suspensions can be utilized by for example, intramuscular, intrathecal, epidural, intraperitoneal, intravenous, subcutaneous, intracerebral or intracerebroventricular injection. The inhibitor may be prepared as a sterile solid composition that may be dissolved or suspended at the time of administration using sterile water, saline, or other appropriate sterile injectable medium. Vehicles are intended to include, where appropriate, inert binders, suspending agents, lubricants, flavourants, sweeteners, preservatives, dyes, and coatings.

Although a preferred use of the inhibitors of the invention is the treatment of cancer, macular degeneration, psoriasis, arthritis, multiple sclerosis and atherosclerosis, said inhibitors of the inventions can be use in the treatment of angiogenesis itself or in the treatment of any disease wherein angiogenesis may play an important role. Examples of such diseases include, but are not limited to diseases involving infection by organisms such as pneumocystis carinii, trypsanoma cruzi, trypsanoma brucei, crithidia fusiculata, as well as parasitic diseases such as schistosomiasis and malaria, tumours (tumour invasion and tumour metastasis), and other diseases such as metachromatic leukodystrophy, muscular dystrophy, amytrophy and similar diseases, osteoporosis, gingival diseases such as gingivitis and periodontitis, Paget's disease, hypercalcemia of malignancy, e.g. tumour-induced hypercalcemia and metabolic bone disease, osteoarthritis, rheumatoid arthritis, atherosclerosis (including atherosclerotic plaque rupture and destabilization), autoimmune diseases, respiratory diseases and immunologically mediated diseases (including transplant rejection), asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), acute/adult respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity particularly as consequent to other drug therapy, in particular other inhaled drug therapy, eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction. Beside cancer, particularly preferred diseases are angiogenesis/vascular endothelium-related disease including macular degeneration, psoriasis, arthritis, multiple sclerosis and atherosclerosis.

The term “cancer” includes for example, melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, lymphoma, brain, colon or bladder cancers. In a most preferred embodiment,

In still more preferred embodiments said angiogenesis-related diseases is rheumatoid arthritis, inflammatory bowel disease, osteoarthritis, leiomyomas, ademonas, lipomas, hemangiomas, fibromas, vascular occlusion, restenosis, atherosclerosis, pre-neoplastic lesions, carcinoma in situ, oral hairy leukoplakia or psoriasis may be the subject of treatment. In a most preferred embodiment, the diseases to be treated by the compounds of the invention are psoriasis, arthritis, multiple sclerosis and atherosclerosis, in particular rheumatoid arthritis. In another preferred embodiments, the cancer involves a tumor, which may or may not be resectable. Moreover, the cancer may involve metastatic tumor(s) or a tumor possibly capable of metastasis.

Cancer cells that may be treated by methods and compositions of the invention also include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating scierosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignit; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In addition in another embodiment of the invention illustrated in the examples, the present inventors provide a GPR4 knock-out mouse. It is to be understood that GPR4 knock-out mice are only a preferred embodiment of a non human mammal wherein the gene coding for GPR4 has been deleted. Hence, a specific reference to a knock-out mouse is intended to be solely exemplary and is intended to refer to any such non human mammal wherein the gene coding for GPR4 has been deleted. Such non human mammal wherein the gene coding for GPR4 has been deleted can be used as an experimental model for angiogenesis, cancer or arthritis, and for screening for compounds modulating angiogenesis, cancer or arthritis

All of the features described herein (including any accompanying claims, abstract and drawings), and/or all of the steps of any method or process so disclosed, may be combined with any of the above aspects in any combination, except combinations where at least some of such features and/or steps are mutually exclusive.

### 6. EXAMPLES

Materials and Methods

Bioinformatics Screen

Seven marker genes (official human gene symbols: KDR, TIE1, TEK, ANGPT2, CDH5, VWF, PTPRB) known to be expressed almost exclusively in endothelial cells and to show similar expression profiles were selected as reference.

Data from more than 100 microarray experiments (Affymetrix HG-U133A) done with cell lines, primary cells and various human tissues were analyzed using GeneSpring (gene expression software provided by Silicon Genetics®). For each of the 7 marker genes all genes with a similar expression profile were determined using the Pearson correlation. Only genes with a correlation coefficient of 0.7 or above to at least one of the 7 marker genes were considered.

RNA Extraction and RT-PCR

Total RNA was recovered from cells using the QIAGEN RNA-easy Mini kit. Prior to reverse transcription, RNAs were treated with DNase I according to manufacturer's procedure (Amplification Grade DNaseI, Invitrogen, Basel, Switzerland). Reverse transcription was performed in a total volume of 30 μl. The reaction mix contained: 20 μl of 2 μg DNaseI-treated RNA, 2 μl of 10× Buffer RT, 2 μl dNTP mix (5 mM each dNTP), 2 μl oligo-dT primer (10 uM), 1 ml RNAse inhibitor (10 U/ul), 1 μl Omniscript reverse transcriptase (QIAGEN, Basel, Switzerland), 2 μl RNAse-free water. The mixture was incubated for 60 minutes at 37° C., then the reaction was heat-inactivated at 93° C. for 5 minutes and then rapidly cooled down on ice. PCR was performed with 5 ml of cDNA and using the Hotstart Mastermix kit (QIAGEN, Basel, Switzerland) Amplification was with the following program: initial denaturation at 95° C. for 15 minutes followed by 45 cycles of 15 s at 94° C., 30 s at 50° C. and 30 s at 72° C. Follow final cycle, melting curve analysis was performed for all tested using ABI7000 software. Primers used were: mouse GPR4-1466F (TGTGCTACCGTGGCATCCT, SEQ.I.D.NO:21), Mouse GPR4-1581R (AAAGCACACCAGCACAATGG, SEQ.I.D.NO:22), mouse GAPDH-F960 (TTGTCAAG CTCATTTCCTGGTATG, SEQ.I.D. NO:23) mouse GAPDH-1062R (TGGTCCAGGGTTTCTTACTCCTT, SEQ.I.D. NO:24); human GPR4-2319F (TGTGCTACCGTGGCATCCT, SEQ.I.D. NO:25), human GPR4-2469R (CTTGAGTTC TGACATTCTCCCTCTT, SEQ.I.D.NO:26); human GAPDH-111F (CAGGGCTGCTTTTAACTCTGGTA, SEQ.I.D. NO:27); human GAPDH-211R (GGGTGGAATCATATTGGAACATG, SEQ.I.D. NO:28).

Generation of Stable HeLa-GPR4 Cells

For the generation of the stable Hela GPR4 cell line the pcDNA3.1(+)/myc-His vector (Invitrogen, Basel, Switzerland) expressing full length human GPR4 was linearized with PvuI and transfected using Effectene reagent (QIAGEN, Basel, Switzerland). Stable cell clones expressing the receptor were isolated following selection in the culture medium at pH 7.4, without Hepes and with antibiotic G418 (400 pg/ml). After 18 days, cells were grown further at pH 7.9.

Cell Culture

Hela-GPR4 stable cells were grown in a 1:1 mixture of bicarbonate-buffered DMEM and Ham's F12 medium supplemented with 10 mM Hepes, 10% foetal calf serum and antibiotics at pH 7.9.

HUVEC cells were purchased from Promo Cell (BioConcept AG, Allschwil, Switzerland, C-10251), and cultured in Medium C-22210 plus Supplement kit C-39210 (both from Promocell/BioConcept AG, Allschwil, Switzerland) and a final concentration of 5% fetal calf serum (South American10270-106, Gibco/Invitrogen, Basel, Switzerland). Mouse lung endothelial cells were isolated and cultured according to the protocol described by Reynolds et al¹³ with the modification that the positive sort was done with a 1:1 mixture of rat anti-mouse VE-cadherin (clone 11D4.1) and anti-CD31 (clone MEC13.3; both from Becton Dickinson, Allschwil, Switzerland).

cAMP Formation Assay

Confluent cell cultures grown in 24 well plates were labelled with [³H]adenine (100 MBq/ml; Amersham, Zürich, Switzerland) for 4 h in serum-free DMEM medium. Cells were then incubated at 37° C. in buffered salt solution containing 130 mM NaCl, 0.9 mM NaH₂PO₄, 5.4 mM KCl, 0.8 mM MgSO₄, 1.0 mM CaCl₂, 25 mM glucose, 20 mM Hepes. The pH of all solutions was adjusted to the indicated value at room temperature. The phosphodiesterase inhibitor isobutylmethylxanthine (IBMX, 1 mM), was added as indicated to allow accumulation of cAMP. Forskolin (FSK) activates adenylyl cyclases in synergy with G_{□s} stimulations and was therefore used to increase the assay window. Incubation time was 15 minutes. Cells were then extracted with ice-cold trichloroacetic acid and cAMP separated from free adenine and ATP using batch column chromatography according to the method described by Salomon¹⁴.

siRNA

All siRNAs were designed using our proprietary algorithm described in WO 2005/059132 (Novartis Nucleic Acid Science unit, Basel, Switzerland), and were synthesized by QIAGEN. A standardized mRNA fusion-construct assay was used to screen several different siRNAs for their potency in targeting human and mouse GPR4, respectively¹⁵. The most potent siRNAs were used in this study. Lyophilized siRNAs were resuspended in the provided hybridization buffer prior usage. As a control siRNA (siCtrl) the non-targeted siRNA from QIAGEN was used.

HUVEC cells (passage 3) were transfected with Hiperpefect (QIAGEN) according to manufacturer's instructions, 3 μl of Hiperfect transfection reagent were used for 30,000 cells in a 24-well and the final siRNA concentration was 10 nM. RNA was harvested 48 h after transfection using QIAGEN RNeasy kit and following the manufacturer's instructions.

Generation of pRAY2-GPR4 Targeting Vector

Arms of homology were amplified by Polymerase Chain Reaction (PCR) from SV129 genomic DNA with the KOD HIFI DNA polymerase (Novagen). Primers were designed according to the sequence of the mouse GPR4 gene (mCG50351.1). The 5′ arm was amplified using sense primer CTGGCCATACTGGCCGGATGTGGCTCAGTTGTTAC (SEQ.I.D.NO:29) and antisense primer CCGCTCGAGTCATGCTTATACCAGCGGTGTCATGCTTAT (SEQ.I.D. NO:30, product size 2.0 kb). The 3′arm was amplified with primer sense CCATCGATGGCTGGCAGATAAG GACAGACG (SEQ.I.D. NO:31) and primer antisense ATAAGAATGCGGCCGCAGCCTCTTCAGTGA CTATCC (SEQ.I.D.NO:32, product size 1.5 kb). The resulting 5′ and 3′ arms were cloned in the pRAY2 vector (Genbank accession number U63120). All PCR fragments and the resulting vectors were sequence verified.

Generation of GPR4 Knock Out Mice

Twenty μg of SfiI linearized targeting vector (pRAY2-GPR4) were electroporated into 1.5×10⁷ BALB/c cells¹⁶, which were subsequently cultured in the presence of 0.2 mg/ml G418 on mitotically inactivated mouse embryonic fibroblasts. The targeted mutation was identified by PCR using primers P1 TGATATTGCTGAAGAGCTTGGCGGC (SEQ.I.D. NO:33) (in Neo gene) and P2 CACTTCCTCTCCCTCCTATTTG (SEQ.I.D. NO:34) followed by a nested PCR with primers P3 AGCGCATCGCCTTCTATCGCC (SEQ.I.D. NO:35) (in Neo gene) and P4 CCAGCACTGTAAGACCTTC (SEQ.I.D. NO:36) (FIG. 3). Homologous recombination was successfully confirmed by Southern blot analysis with an external 5′ probe (PCR product from primers cgtgcttgttaagcgaatac (SEQ.I.D. NO:37) and agtcattccagaagcctaga (SEQ.I.D. NO:38) on SacI, BamHI, EcorV or HindIII digested genomic DNA. The neomycin probe (1.2 kb fragment from a BamHI NheI digested pRAY2 vector) revealed a single integration site. Subsequently, positive ES cell clones were microinjected into C57BL/6 blastocysts and re-implanted in pseudo-pregnant foster mothers. The resulting male chimeras were crossed with Balb/c females and germ line integration was determined using fur colour, PCR and Southern blot hybridization. Breeding of these GPR4 heterozygote mice was successful in obtaining homozygous GPR4 knockout mice in a mendelian ratio. Absence of GPR4 transcripts was evaluated on heart, kidney, liver, lung, spleen and testis cDNA from GPR4-deficient and wild type mice. Mice were euthanized by CO₂ inhalation and tissues were quickly removed and stored in RNA later (Ambion, Huntingdon, UK). RNA was prepared with the Absolutely RNA RT-PCR miniprep kit (Stratagene, Amsterdam, The Netherlands) and reverse transcribed with the Omniscript kit (QIAGEN, Basel, Switzerland). Multiplex PCR was performed with primers GCTGCCATGTGGACTCTCGA (SEQ.I.D.NO:39) and CAGGAAGGCGATGCTGATAT (SEQ.I.D. NO:40) for GPR4 and gctcacatgggaatgttcac (SEQ.I.D. NO:41) and atgttgtcaaagttgtcataag (SEQ.I.D. NO:42) for Clathrin-2K as control. Mice had unrestricted access to food and water and all procedures were carried out in accordance with the Swiss law for animal protection.

Animals

Female Balb/C mice (WT or GPR4 KO) of 6-8 weeks of age were bred at the Novartis animal breeding facility. Control Balb/C mice were obtained from Charles River Laboratories (Les Oncins, France). Mice were identified via ear markings and kept in groups (5-6 animals per cage) under normal conditions and observed daily. Five to ten mice were used per treatment group and all animal experiments were performed in strict adherence to the Swiss law for animal protection. All animal experiments were performed at least twice.

Angiogenesis Growth Factor Implant Model

The model has been previously described¹⁷. Briefly, porous tissue chambers made of perfluoro-alkoxy-Teflon (Teflonâ-PFA, 21 mm×8 mm diameter, 550 μl volume) were filled with 0.8% agar (BBLâ Nr. 11849, Becton Dickinson, Meylan, France) and 20 U/ml heparin, (Roche, Basel, Switzerland) supplemented with or without 3 mg/ml recombinant human VEGF165¹⁸ and 0.3 mg/ml bFGF (Invitrogen, Basel, Switzerland) and siRNAs as indicated. Solutions were maintained at 39° C. prior the filling procedure. Mice were anesthetized using 3% Isoflurane (Forenea, Abbott AG, Cham, Switzerland) inhalation. For subcutaneous implantation, a small skin incision was made at the base of the tail to allow the insertion of an implant trocar. The chamber was implanted under aseptic conditions through the small incision onto the back of the animal. The skin incision was closed by wound clips (Autoclip 9 mm Clay Adams). On the 4th day after implantation, animals were sacrificed using CO₂. For the siRNA experiments, siRNA were added at a final concentration of 0.3 mM together with the growth factors into the chambers and animals were sacrificed 3 days after implantation. Chambers were excised and the vascularized fibrous tissue formed around each implant carefully removed and weighed. Body weight was used to monitor the general condition of the mice.

Quantification of the Angiogenic Response

The fibrous tissue that grew around the implant was homogenized for 30 seconds at 24,000 rpm (Ultra Turrax T25) after addition of 1 ml RIPA buffer (50 mM Tris-HCL pH7.2, 120 mM NaCl, 1 mM EDTA pH8.0, 6 mM EGTA pH8.5, 1% NP-40, 20 mM NaF) to which 1 mM Pefabloc SC Proteinase inhibitor (Roche, Basel, Switzerland) and 1 mM Na-Vanadate were freshly added. The homogenate was centrifuged for 30 min at 7000 rpm and the supernatant was filtered using a 0.45 μm GHP syringe filter (Acrodisca GF, Gelman Sciences, Ann Arbor, Mich.) to avoid fat contamination. This lysate was used for measuring Tie2 protein levels by ELISA as described¹⁹.

Orthotopic Tumour Models

4T1 mouse breast cancer cell lines were obtained from ATCC (LGC Promochem, Molsheim, France) and grown in DMEM high glucose+1% Glutamine, +10% FCS. CT26 cells were obtained from ATCC and grown in MEM+10% FCS+1% sodium pyruvate +1% Glutamine+1% non-essential amino acids+2% Vitamins. Under light isoflurane anesthesia, 20 μL of 4T1 cell suspension (5×10⁷ cells/ml in PBS) were injected under the fat pad of the 4th mammary gland, to give a total inoculum of 10⁶ cells per mouse.

For the CT26 tumour model, cell inoculation was performed in an OHC zone, under aseptic conditions. Prior to surgery, the mice are anaesthetized with a single subcutaneous injection of a freshly prepared mixture of ketamine (100 mg/kg) and xylazine (5 mg/kg). The abdomen (skin and muscle) of was opened with a surgical scissor along the linea alba (0.5-1 cm long). With neutral forceps, the caecum of the animal was located, and gently pulled out from the abdomen. With a 30 Gauge needle (Becton-Dickinson, 320834), 10 ml of a CT26 cell suspension (10⁶ cells/ml in HBSS) was injected in the submucosa layer with the assistance of a magnifying glass, to give a total inoculum of 1×10⁵ cells per mouse. Finally, the caecum was placed back into the abdomen, the muscle was sutured (Dexon, 9104-11), and the wound was closed with 3 Autoclips® (Clay-Adams 427631). Animals were finally transferred to a 37° C. heating pad to recover from the anesthesia. Animals were sacrificed 19-21 days after tumour implantation. Body weight was used to monitor the general condition of the mice.

Immunofluorescence and Vessel Counts

8 mm cryosections sections of CT26 orthotopic tumours were fixed with acetone, blocked with 10% normal goat serum (NGS) and incubated with primary antibodies diluted 1:200 in PBS/0.5% NGS/0.05% TritonX-100for 2 h at room temperature. Sections were washed 3 times with PBS and incubated with the secondary antibodies diluted 1:400 in PBS/0.5% NGS/0.05% TritonX-100. After 1 h incubation at room temperature, sections were washed with PBS and mounted in Mowiol. Antibodies used were rat anti-mouse CD31 (clone MEC13.3, BD PharMingen, San Diego, Calif.) and rabbit anti mouse Ki67 (Neomarkers, Fremont, Calif.). Secondary antibodies were goat anti-rabbit ALEXA Fluor 568 and goat anti-rat ALEXA Fluor 488 (both from Molecular Probes, Invitrogen, Basel Switzerland). Ki67 stainings were used to quantify the amount of proliferative cells in CT26 tumours. Six to eight representative pictures were taken from each tumour (n=6 per group) using a Zeiss Axioplan microscope (20× lens). Percent coverage of proliferative cells of the total area were calculated by using a Microsoft program.

To determine vessel density, vessels were stained for CD31 as described above were counted manually over the whole tumour section. Pictures encompassing the whole tumour where taken at 10× magnification using a Zeiss Axioplan microscope. The area of the counted regions was measured using the Openlab 3.1.5 software (Improvision, Lexington, Mass.). Six complete tumours were counted per group.

FACS Analysis

Non-transfected and transfected HUVEC cells were analyzed by FACS for VEGFR2 levels. Briefly, cells were trypsinized, washed with PBS+10% FCS and incubated 10 minutes on ice prior to the addition of RPE-conjugated mouse anti human VEGFR2 mAb (1 mg/10⁶ cells; R&D Sytems, Abingdon, UK). RPE-labeled isotype mouse IgG1 was used as FACS control (R&D systems, Abingdon, UK). FACS analysis was performed on a FACScalibur using Cell Quest Software (Becton-Dickinson, Allschwil, Switzerland).

SDS-PAGE, Western-Blot, ELISA

Total protein was extracted from tissues with RIPA buffer supplemented with protease inhibitor (Complete, Roche Diagnostics, Switzerland). Proteins were resolved on 8% SDS-PAGE, then blotted onto PVDF membrane and probed with different antibodies (rat anti-mouse VE-Cadherin mAb, clone 11D4.1, Becton Dickinson, Allschwil, Switzerland; goat anti-mouse EphB4, R&D systems, Abingdon, UK; rabbit anti mouse tubulin, Spring Biosciences, Freemoiont. Calif.). Detection was performed with HRP-labeled secondary antibodies and ECL-plus chemioluminescent reagent (Amersham Biosciences, Uppsala, Sweden). Level of Tie2 receptor was determined using a Tie2 ELISA as described ¹⁹. Mouse VEGFR2 levels were measured using a commercially available ELISA kit (R&D systems, Abingdon, UK).

Results

GPR4 is Expressed in Endothelial Cells

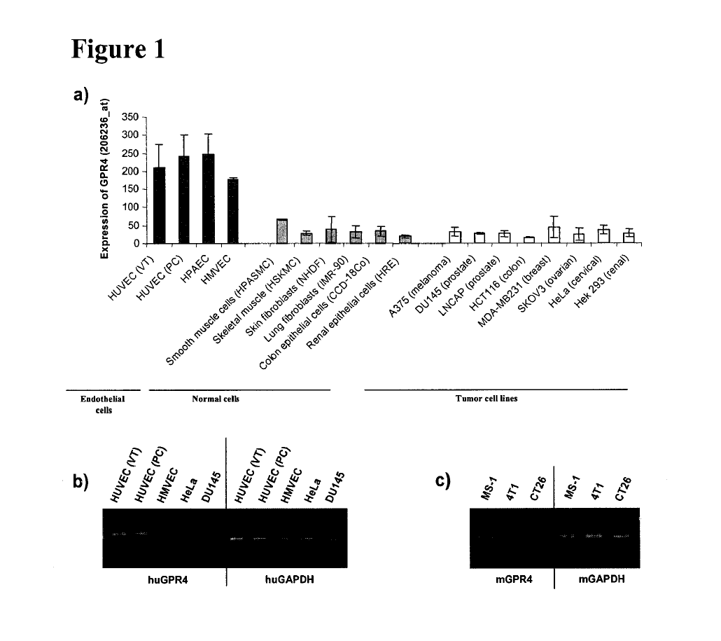

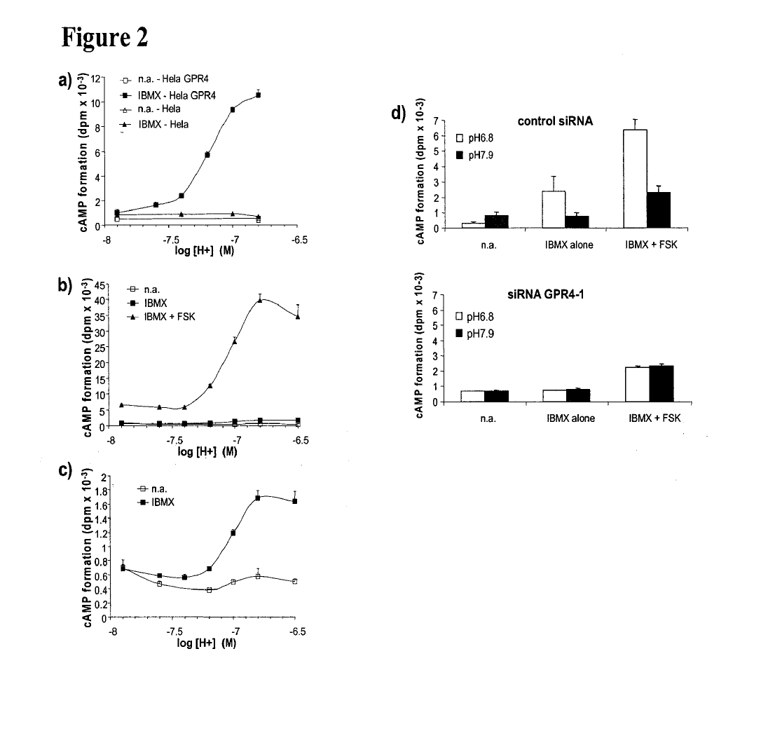

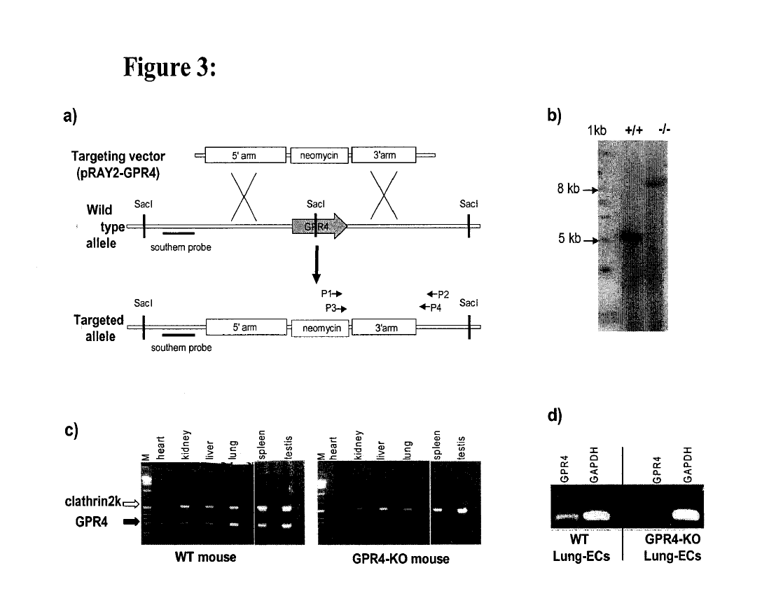

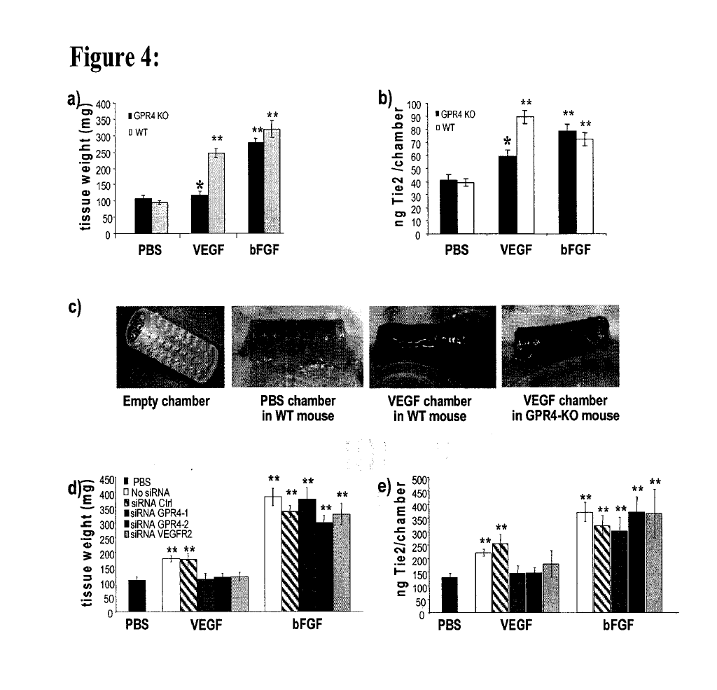

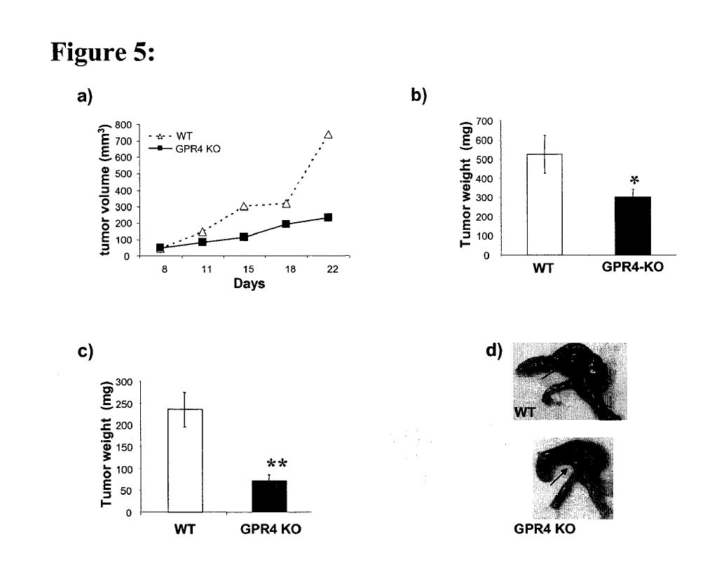

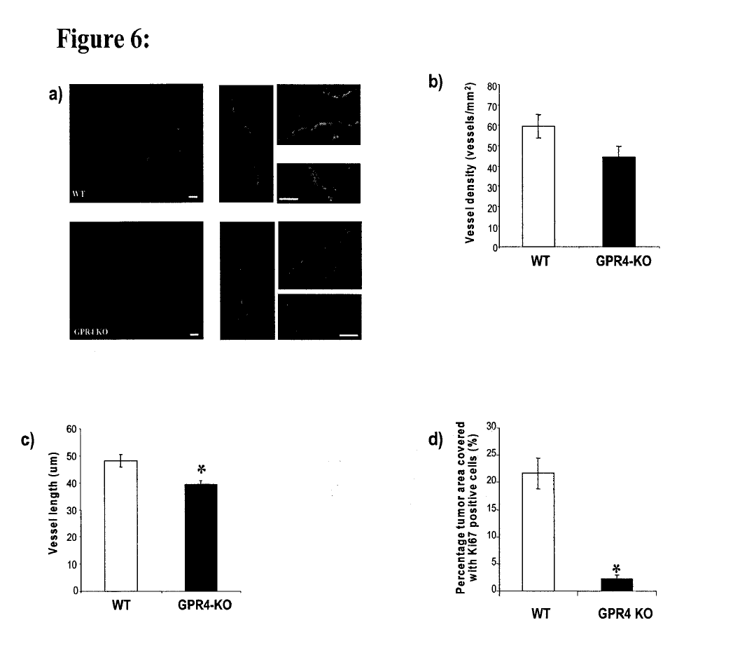

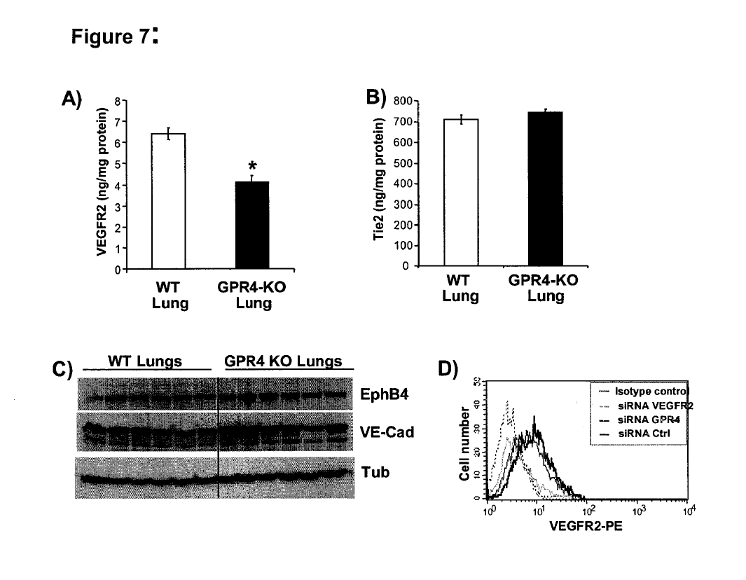

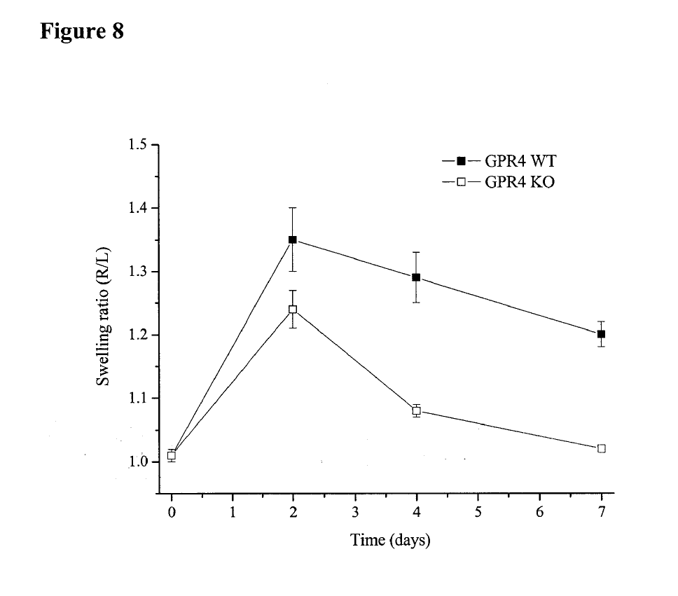

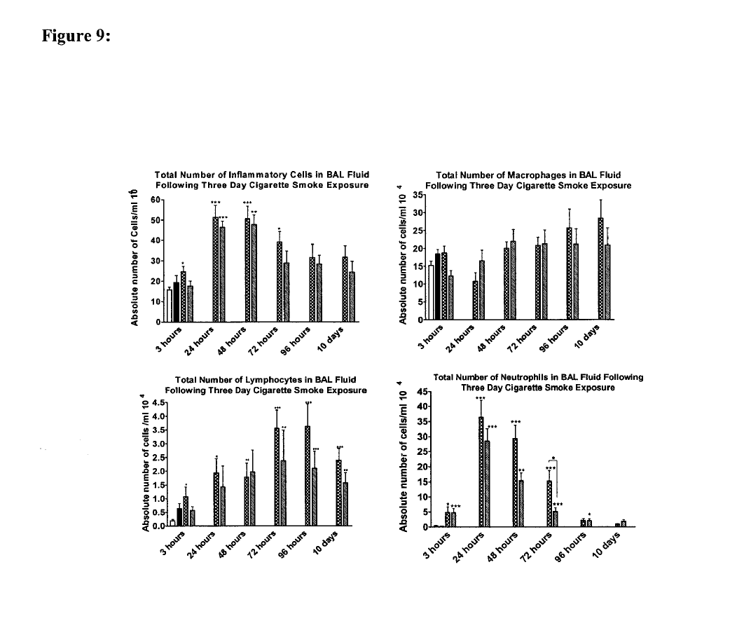

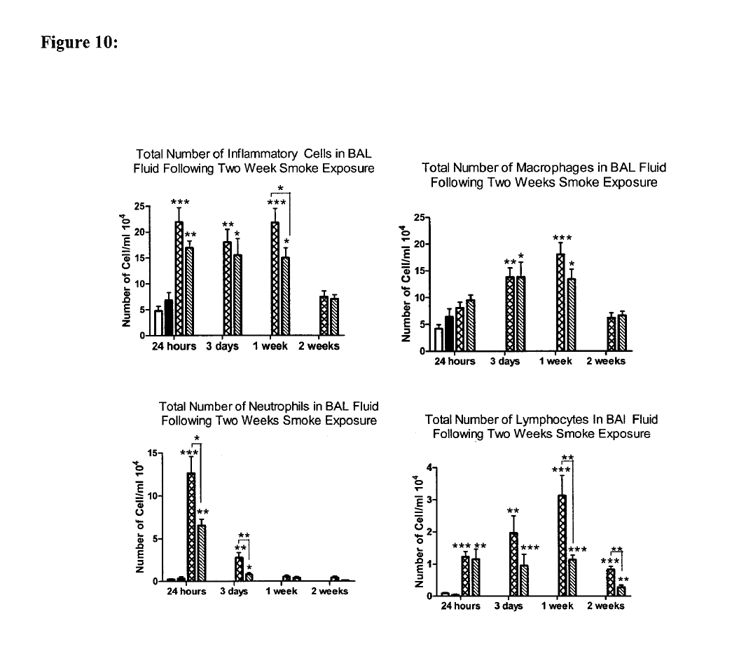

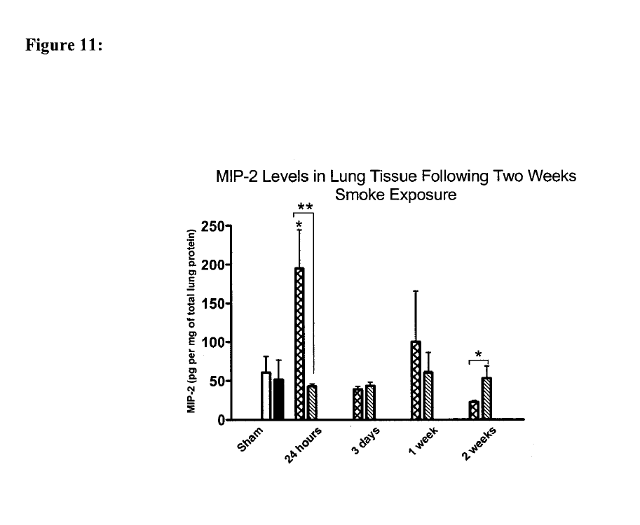

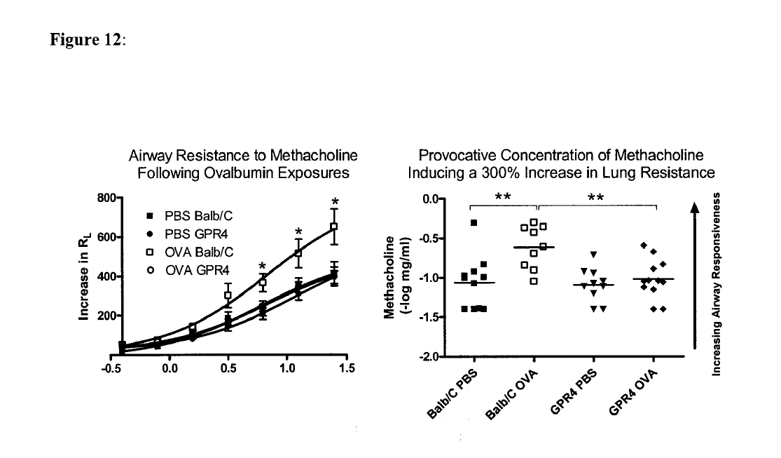

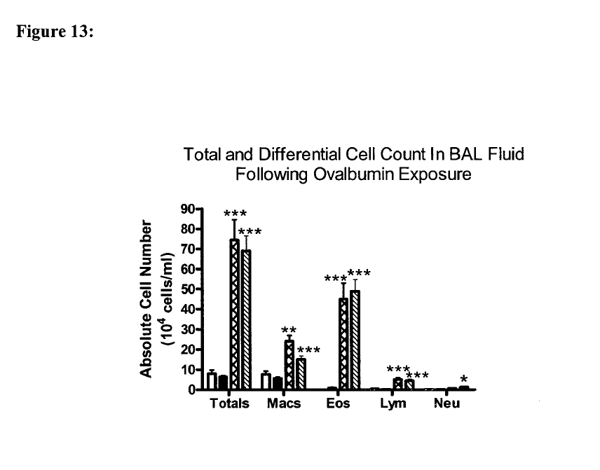

Analysis of gene expression data obtained using DNA microarrays of various cell types and tissues of human origin showed that expression of GPR4 correlated with the expression of a set of marker genes characteristic for endothelial cells (not shown). We could confirm specific expression of GPR4 in endothelial cells by extended DNA microarray analysis (FIG. 1a) as well as by RT-PCR in human and mouse endothelial cells (FIG. 1b-c).

GPR4 Acts as a Functional Proton-Sensing Receptor in Endothelial Cells

To demonstrate that GPR4 is a functional pH sensor on endothelial cells we exposed HUVECs (primary human umbilical vein endothelial cells) to mild extracellular acidosis and measured cAMP production. HeLa cells transfected with recombinant GPR4 were used as controls (FIG. 2a). HUVECs showed a weak but clearly significant response, and this response could be strongly amplified by the addition of forskolin (FIG. 2b,c). The maximal response was observed around pH 6.8 and there was no significant cAMP production at pH 7.9 in all GPR4 expressing cells, in agreement with our previous results¹. To demonstrate that GPR4 is the pH sensor responsible for this increased cAMP production, we transfected HUVECs with siRNAs 48 h prior to exposure to extracellular acidosis. A GPR4-specific siRNA was able to abrogate the pH-dependent cAMP increase, whereas control siRNA had no effect (FIG. 2d). The response to acidosis could also be inhibited by specific low molecular weight antagonists of the GPR4 receptor (data not shown).

GPR4-Deficient Mice are Viable and Fertile

In order to better understand the function of GPR4, GPR4-deficient mice were generated by replacing the coding sequence of the receptor with a neomycin resistance cassette (FIG. 3a). Correct targeting of the GPR4 gene was verified by Southern blotting (FIG. 3b). Expression of GPR4 mRNA was also measured by RT-PCR in several organs as well as primary lung endothelial cells isolated from both wild type and GPR4-deficient mice. As expected, GPR4 mRNA was absent in all tissues from the GPR4-deficient mice but present in the wild type controls (FIG. 3c). FIG. 3d shows GPR4 expression in primary endothelial cells isolated from lungs of wild type mice, further confirming that GPR4 is expressed in endothelial cells, but not in cells from GPR4-deficient mice. GPR4-deficient mice are viable and fertile and show no gross abnormalities compared to their wild type littermates, demonstrating that GPR4 is not essential during development. In addition, no significant histopathological differences were evident in the GPR4-deficient mice when compared to age- and gender-matched wild type animals. Notably, the cardiovascular system appeared normal. Slight differences in organ weight were noted for lungs, ovaries and testes when comparing GPR4-deficient mice to wild type. However, considering the absence of corroborative histopathological findings, these organ weight changes may be incidental.

Impaired VEGF-Driven Angiogenesis in GPR4-Deficient Mice

To investigate whether GPR4 plays a role in pathological angiogenesis, GPR4-deficient mice were subjected to a growth factor implant angiogenesis model^{17,19}. For this purpose, mice were implanted with Teflon chambers containing either VEGF or bFGF (basic fibroblast growth factor), two well known angiogenic factors, or PBS as a baseline control. The addition of an angiogenic factor triggers the formation of a new, well vascularized tissue around the implanted chamber. As shown in FIG. 4a-c, GPR4-deficient mice failed to show an angiogenic response to VEGF, whereas the response to bFGF was similar to that observed in wild type controls. In order to confirm this striking effect, we added GPR4-specific siRNA together with the different growth factors into the implanted chambers. This type of local delivery of siRNAs was used before to downregulate angiogenesis targets (E.B manuscript in preparation;²⁰). In agreement with the previous experiment, two independent GPR4-specific siRNAs abrogated the angiogenic effect of VEGF, but had no effect if combined with bFGF (FIG. 4d). A similar effect was observed with an siRNA specific for VEGFR2, whereas a control siRNA had no effect with either growth factor.

Reduced Tumour Growth in GPR4-Deficient Mice

Since angiogenesis is crucial for tumours, we next investigated whether lack of GPR4 in the host would affect tumour growth. Syngeneic 4T1 breast tumour cells were implanted into the fat pad of GPR4-deficient or wild type female mice. Tumour growth was monitored over 3 weeks by caliper measurement and tumours were weighed at the end of the experiment. As shown in FIGS. 5a and 5b, tumours were markedly smaller in GPR4-deficient mice as compared to wild type controls. Injection of syngeneic CT26 colon tumour cells was used as a second tumour model. Cells were implanted orthotopically into the caecum of mice, and tumour weight was measured after 20 days. In this model the reduction in tumour growth in GPR4-deficient mice compared to wild type controls was even more pronounced (FIG. 5c, d).

Reduced Vascularity and Reduced Cell Proliferation in Tumours Grown in GPR4-Deficient Mice

CT26 tumours were analyzed histologically in more detail (FIG. 6a-6d). When tumour sections were stained for CD31, a pan-endothelial marker, we noted that the endothelial cells looked frail and disrupted and vessels appeared not correctly shaped (FIG. 6a). When CD31 stained vessels were quantified, we observed a reduction in vessel density in the tumours grown in the GPR4-deficient mice. Unfortunately, due to the high variability and the morphological differences of the vessels, this difference did not reach statistical significance (P=0.11; FIG. 6b). Staining for the nuclear proliferation antigen Ki67 revealed a highly significant reduction of proliferating cells in tumours grown in GPR4-deficient mice (FIG. 6c) compared to wild type controls. In contrast, we saw no difference in staining for apoptosis (activated Caspase3) or smooth muscle cells/pericytes (smooth muscle actin, desmin, NG2) between tumours grown in wild type and in GPR4-deficient mice (data not shown).

GPR4 Deficiency Results in Decreased VEGFR2 Levels

Trying to understand why GPR4-deficient mice are specifically refractory to VEGF-driven angiogenesis, we investigated the expression of VEGFR2, the main signaling receptor for VEGF on endothelial cells, in wild type and GPR4-deficient mice. Interestingly, we found decreased levels of VEGFR2 in lungs and kidneys of GPR4-deficient mice (FIG. 7a and data not shown). This difference was not due to a difference in endothelial cell numbers, since the levels of other endothelial markers such as Tie2, VE-cadherin and EphB4 were similar between wild type and GPR4-deficient mice (FIG. 7b,c). Furthermore, when GPR4-specific siRNAs were transfected into HUVECs, a reduction in VEGFR2 surface levels was measured by FACS analysis, which was not seen in HUVECs transfected with a control siRNA (FIG. 7d). We therefore conclude that the reduced angiogenic response to VEGF of GPR4-deficient endothelial cells is due, at least in part, to a decreased expression of VEGFR2.

Mouse Antigen-Induced Arthritis Model

Female GPR4 wild type and GPR4-deficient mice were sensitized i.d. on the back at two sites to methylated bovine serum albumin (mBSA—Fluka Chemie AG) homogenized 1:1 with complete Freund's adjuvant on days -21 and -14 (0.1 ml containing 1 mg/ml mBSA). On day 0, the right knee received 10 ml of 10 mg/ml mBSA in 5% glucose solution (antigen injected knee), while the left knee received 10 μl of 5% glucose solution alone (vehicle injected knee). The diameters of the left and right knees were then measured using callipers immediately after the intra-articular injections and again on days 2, 4 and 7. Right knee swelling was calculated as a ratio of left knee swelling, and the R/L knee swelling ratio plotted against time to give Area Under the Curve (AUC) graphs for control and treatment groups. The percentage inhibition of the individual treatment group AUCs were calculated vs the control group AUC (0% inhibition) using an Excel spreadsheet. On day 7, the mice were killed by CO₂ inhalation and the right and left knees removed and processed for histological analysis. Knees were processed for undecalcified histology using a Histodur plastic embedding method (Leica AG, Germany). Sections (5 μm) from both the control and arthritic knees were cut on a RM 2165 rotation microtome (Leica AG, Germany). After Giemsa staining, according to standard protocols, the slides were number coded as left knee/right knee pairs from each animal and read in a blinded fashion.

Reduced Severity of Arthritis in GPR4-Deficient Mice

Angiogenesis plays an important role in inflammatory arthritis by controlling the growth of synovial pannus. Proliferating pannus tissue composed mainly of synovial fibroblasts and macrophages is responsible for the destruction of cartilage and subchondral bone. In a similar way to solid tumour growth, the invading pannus tissue cannot proceed beyond a certain point without an adequate blood supply. Angiogenesis inhibitors are effective in arthritis models.

In GPR4-deficient mice there was a marked and significant inhibition of knee swelling compared to wild type mice as shown in FIG. 8. with a reduction in the swelling AUC_{[0-7days]} of 58.7%.

GPR4 and COPD

Methods

Animal Maintenance and Statement of Welfare

Balb/C and GPR4−/− mice (male and female) (20-28 g) (Charles River, Margate, UK) were housed in rooms maintained at constant temperature (21±2° C.) and humidity (55±15%) with a 12 h light cycle and 15-20 air changes per hour. Animals were allowed food, RMI3 Pellets, (SDS UK Ltd.) and water ad libitum. Studies described herein were performed under a Project License issued by the United Kingdom Home Office and protocols were approved by the Local Ethical Review Process at Novartis Institutes for BioMedical Research, Horsham.

Cigarette Smoke Exposure

Mice were placed in a 7 liter Perspex chamber and cigarette smoke was delivered every 60 seconds with fresh air being pumped in for the remaining time. The smoke was generated using 1 R3F Research Cigarettes (University of Kentucky, Louisville, Ky.) and was drawn into the chambers via a peristaltic pump. Sham, age- and sex-matched control animals were exposed to air only in the same manner for the same duration of time (approximately 55 minutes per exposure period).

Acute Cigarette Smoke Exposure

Animals were exposed to 5 cigarettes per exposure period for three consecutive days. Animals were sacrificed following cigarette smoke exposure with an overdose of terminal anaesthetic (sodium pentobarbitone 200 mg i.p.) followed by exsanguination. Mice were culled at 3 hours, 24 hours, 48 hours, 72 hours, 96 hours and 10 days after the last exposure. Sham-exposed control mice were also culled at each time point.

Sub-Chronic Cigarette Smoke Exposure

Briefly, mice were exposed to 5 cigarettes per exposure period for two weeks as described above. Animals were sacrificed with an overdose of terminal anaesthetic (sodium pentobarbitone 200 mg i.p.) followed by exsanguination 24 hours, 3 days, 1 week and 2 weeks after the last exposure. Sham-exposed control mice were also culled at each time point.

Ovalbumin Exposure

Mice were sensitized on day 0 with an intraperitoneal injection containing 10 μg ovalbumin in 0.2 ml Alum. On Day 14, the mice were given an intraperitoneal booster injection of the same antigen/alum mix. Seven days following the second sensitization, mice were exposed to aerosolised ovalbumin (50 mg/ml in PBS) or PBS aerosol. Animals were exposed to allergen for twenty minutes, twice a day leaving six hours between each exposure. Animals were exposed to, ovalbumin or PBS for three consecutive days, totalling six challenges. Eighteen to twenty fours hours after the last aerosol challenge, mice were prepared for the assessment of bronchial responsiveness to methacholine and bronchoalveolar lavavge fluid and lung tissue was collected.

Measurement of Bronchial Hyper Responsiveness

Eighteen to twenty four hours following allergen exposure animals were anesthetized and tracheostomised with a Portex cannula (1.3 mm cannula inserted into a 1.7 mm cannula, 15 mm length) and ventilated at a rate of 250 strokes/min, with a tidal volume of 250 μl Airflow was monitored with a pneumotachograph connected to a flow transducer (Buxco, UK). Trans-pulmonary pressure was assessed via an oesophageal catheter connected to a pressure transducer (Buxco, UK). The signals from the transducers were digitized using an amplifier interface unit connected to a Microsoft computer and analyzed with Buxco XA software. Buxco XA software has been programmed to instantaneously calculate pulmonary resistance (R_{L}) from airflow and trans-pulmonary pressure measurements. A stable baseline R_{L} was recorded in response to PBS. Increasing doses of methacholine bromide (MCh) aerosol (0.35, 0.7, 1.5, 3, 6, 12.5, 25 mg/ml) were then administered for 10 seconds using a Aeroneb micropump nebulizer (Aerogen, Ireland), and R_{L} was recorded for a 5 minute period. Each dose of MCh was separated by 10 minute intervals with hyperinflation of the airways to twice the tidal volume, by blocking the outflow of the ventilator manually. Hyperinflation was performed in order to ensure constant volume history prior to subsequent MCh exposures. The average R_{L} values during the 5 minute period were expressed as percentage change from baseline after PBS aerosol. The concentration of MCh needed to increase R_{L} by 300% above PBS baseline (PC₃₀₀) was calculated by interpolation of the log concentration-lung resistance curve from individual animals, and log PC₃₀₀₀ was taken as a measure of bronchial responsiveness.

Bronchoalveolar Lavage (BAL) Fluid Inflammation

In all experiments performed lungs were lavaged using a butterfly cannula inserted into the trachea and instilling the lungs with 3×0.4 mL aliquots of sterile PBS. The lavage fluid was centrifuged at 1500 rev/min for 15 minutes at 4° C. The supernatant was aliquoted out and stored at −80° C. for cytokine and chemokine assays. The remaining cell pellet was re-suspended in 0.5 ml methyl violet solution. Total cell counts were performed by haemocytometry. Differential cell counts were performed using standard morphological criteria on Hema-Gurr stained cytospins (300 cells/sample) (Merck, Poole, UK). Leukocyte numbers were determined by multiplying the percentage of each leukocyte subpopulation with the total number of cells for each sample and expressed as cells/mL for BAL cells.

Collection and Homogenization of Lung Tissue

Following the BAL procedure, lungs were excised from the thoracic cavity and snap frozen in liquid nitrogen. All lung samples were stored at −80 C. Frozen lung tissues were homogenized using a motorised tissue grinder, containing RIPA buffer (50 mM Tris-HCl pH7.4, 150 mM NaCl, 0.1% SDS, 1% NP40, 0.5% Deoxycholate acid, 1 tablet complete mini cocktail inhibitor (Roche) per 10m1 of buffer). All samples were kept on ice and homogenised until the solution turned clear. Samples were then centrifuged at 13,000 RPM, for 10 minutes and aliquots of lung homogenate were stored at −80° C.

Tissue Cytokine Analysis by ELISA

MIP-2 was measured using ELISA Duo-Sets from R&D Systems (Abingdon, UK). Tissue homogenate protein levels were measured using the BCA Protein Assay Kit (Pierce, Northumberland, UK) and chemokine values were normalized against protein levels for individual homogenate samples.

Statistical Analysis

All data presented as Mean+Standard Error of Mean (SEM). Student's t-test was used comparing all smoke-exposed animals to their time-matched air-exposed controls. Ovalbumin-exposed animals were compared to their PBS-exposed controls.

Results

Effect of Acute Cigarette Smoke Exposure on GPR4−/− Mice

Following acute cigarette smoke exposure animals were sacrificed at various time points and BAL was performed. An increase in BAL fluid neutrophils was observed as early as 3 hours in both GPR4−/− and Balb/C mice exposed to smoke compared to sham controls. BAL fluid neutrophils peaked at 24 hours, in both smoke exposed GPR4−/− and Balb/C mice. Forty eight hours following smoke exposure the number of neutrophils started to resolve in both GPR4−/− and Balb/C smoke exposed mice. Neutrophil resolution was enhanced in smoke exposed GPR4−/− mice compared to smoke exposed Balb/C, as observed at 72 hours following smoke exposure (5.10×10⁴±1.31 versus 15.24×10⁴±3.43 cells/ml; p<0.05) (FIG. 9).

Effect of Sub-Chronic Cigarette Smoke Exposure on GPR4−/− mice

Following sub-chronic cigarette smoke exposure, neutrophils were increased in BAL fluid from Balb/C smoke-exposed mice and GPR4−/− smoke-exposed mice compared to their sham controls. There was a significant reduction in the number of neutrophils in GPR4−/− mice compared to Balb/C mice at 24 hours and 3 days (FIG. 10). Interestingly, the number of lymphocytes were also increased following cigarette exposure in both GPR4−/− and Balb/C mice. However the number of lymphocytes in GPR4−/− BAL fluid were significantly attenuated at 1 week (10.42×10⁴±2.08 versus 3.57×10⁴±0.57 cells/ml; p<0.01) and 2 weeks (2.76×10⁴±0.31 versus 0.97×10⁴±0.2 cells/ml; p<0.01) following smoke exposure compared to Balb/C mice. Although the number of macrophages in BAL fluid were increased following smoke exposure no difference was observed between GPR4−/− and Balb/C mice.

MIP-2 was significantly reduced at 24 hours following smoke exposure in GPR4−/− mice compared to Balb/C mice (42.7±3.0 versus 194.9±50.1 pg/ml; p<0.01). Surprisingly, MIP-2 levels were significantly higher at 2 weeks following smoke exposure in smoke-exposed GPR4−/− mice compared to smoke-exposed Balb/C mice (53.3±15.4 versus 22.6±1.9 pg/ml; p<0.05) (FIG. 11).

Airway Hyperresponsiveness Following Ovalbumin Exposure in Sensitized GPR4−/− Mice

Twenty four hours following ovalbumin-exposure, airway responsiveness was assessed by methacholine challenge. Animals were exposed to increasing doses of methacholine, and changes in lung function were measured. No difference in airway responsiveness was observed between GPR4−/− mice exposed to PBS compared to Balb/C PBS-exposed mice (FIG. 12). Interestingly, GPR4−/− exposed to ovalbumin were significantly less responsive to methacholine compared to ovalbumin-exposed Balb/C mice (PC₃₀₀−1.02±0.07 versus −0.61±0.09 −log mg/ml methacholine; p<0.01).

Following ovalbumin exposure there was a significant increase in macrophages, eosinophils and lymphocytes in both Balb/C and GPR4−/− ovalbumin-exposed mice compared to the PBS-exposed mice controls. No difference in macrophages, eosinophils or lymphocytes was observed between Balb/C ovalbumin-exposed and GPR4−/− ovalbumin-exposed mice (FIG. 13).

Summary

In the experiments conducted to date, it has been demonstrated that GPR4 gene deletion enhances the resolution of neutrophils in BAL fluid following acute, sub-chronic and chronic cigarette smoke exposure. In addition it has been demonstrated that GPR4 deficiency also attenuates ovalbumin induced airway hyperresponsiveness.

### 7. BIBLIOGRAPHICAL INFORMATION

1. Ludwig, M. G. et al. Proton-sensing G-protein-coupled receptors. Nature 425, 93-8 (2003).

2. Ishii, S., Kihara, Y. & Shimizu, T. Identification of T cell death-associated gene 8 (TDAG8) as a novel acid sensing G-protein-coupled receptor. J Biol Chem 280, 9083-7 (2005).

3. Wang, J. Q. et al. TDAG8 is a proton-sensing and psychosine-sensitive G-protein-coupled receptor. J Biol Chem 279, 45626-33 (2004).

4. Kim, K.S. et al. GPR4 plays a critical role in endothelial cell function and mediates the effects of sphingosylphosphorylcholine. Faseb J 19, 819-21 (2005).

5. Lurn, H. et al. Inflammatory stress increases receptor for lysophosphatidylcholine in human microvascular endothelial cells. Am J Physiol Heart Circ Physiol 285, H1786-9 (2003).

6. Bergers, G. & Benjamin, L. E. Tumorigenesis and the angiogenic switch. Nat Rev Cancer 3, 401-10 (2003).

7. Griffiths, J. R. Are cancer cells acidic? Br J Cancer 64, 425-7 (1991).

8. Gatenby, R. A. The potential role of transformation-induced metabolic changes in tumor-host interaction. Cancer Res 55, 4151-6 (1995).

9. Gatenby, R. A. & Gillies, R. J. Why do cancers have high aerobic glycolysis? Nat Rev Cancer 4, 891-9 (2004).

10. Gambhir, S. S. Molecular imaging of cancer with positron emission tomography. Nat Rev Cancer 2, 683-93 (2002).

11. Park, H. J., Lyons, J. C., Ohtsubo, T. & Song, C. W. Acidic environment causes apoptosis by increasing caspase activity. Br J Cancer 80, 1892-7 (1999).

12. Pouyssegur, J., Dayan, F. & Mazure, N. M. Hypoxia signalling in cancer and approaches to enforce tumour regression. Nature 441, 437-43 (2006).

13. Reynolds, L. E. & Hodivala-Dilke, K. M. Primary mouse endothelial cell culture for assays of angiogenesis. Methods Mol Med 120, 503-9 (2006).

14. Salomon, Y. Adenylate cyclase assay. Adv Cyclic Nucleotide Res 10, 35-55 (1979).

15. Husken, D. et al. mRNA fusion constructs serve in a general cell-based assay to profile oligonucleotide activity. Nucleic Acids Res 31, e102 (2003).

16. Gassmann, M. et al. Redistribution of GABAB(1) protein and atypical GABAB responses in GABAB(2)-deficient mice. J Neurosci 24, 6086-97 (2004).

17. Wood, J. et al. Novel antiangiogenic effects of the bisphosphonate compound zoledronic acid. J Pharmacol Exp Ther 302, 1055-61 (2002).

18. Siemeister, G. et al. Expression of biologically active isoforms of the tumor angiogenesis factor VEGF in Escherichia coli. Biochem Biophys Res Commun 222, 249-55 (1996).

19. Ehrbar, M. et al. Cell-demanded liberation of VEGF121 from fibrin implants induces local and controlled blood vessel growth. Circ Res 94, 1124-32 (2004).

20. Chae, S. S., Paik, J. H., Furneaux, H. & Hla, T. Requirement for sphingosine 1-phosphate receptor-1 in tumor angiogenesis demonstrated by in vivo RNA interference. J Clin Invest 114, 1082-9 (2004).

1. Use of a GPR4 inhibitor for the manufacture of a medicament for the inhibition of angiogenesis.

2. Use of claim 1 wherein said inhibition of angiogenesis is for the treatment of cancer, macular degeneration, psoriasis, arthritis, multiple sclerosis or atherosclerosis.

3. Use of claim 1 wherein said inhibitor is a siRNA.

4. Use of claim 3 wherein said siRNA is double-stranded.

5. Use of claim 4 wherein said medicament is for treating a human being and the sequences of said siRNA is:

| | |
|---|---|
| sense: | |
| 5′-GCGCTGTGTCCTATCTCAAdTdT-3′ | (SEQ ID NO: 1) |
| anti-sense: | |
| 5′-TTGAGATAGGACACAGCGCdAdG-3′. | (SEQ ID NO: 2) |

6. Use of claim 4 wherein said medicament is for treating a human being and the sequences of said siRNA is:

| | |
|---|---|
| sense: | |
| 5′-CCATGTCTGGCCAGATAAAdTdT-3′ | (SEQ ID NO: 4) |
| anti-sense: | |
| 5′-TTTATCTGGCCAGACATGGdCdG-3′. | (SEQ ID NO: 5) |

7. Use of claim 4 wherein said medicament is for treating a human being and the sequences of said siRNA is:

| | |
|---|---|
| sense: | |
| 5′-CATAAGACCGCAATTCTAAdTdT-3′ | (SEQ ID NO: 7) |
| anti-sense: | |
| 5′-TTAGAATTGCGGTCTTATGdTdT-3′. | (SEQ ID NO: 8) |

8. A method of treating a subject with a medicament according to claim 1.

9. A siRNA molecule comprising the sequence of SEQ ID NO:1-20.

10. A non human animal wherein the gene coding for GPR4 has been deleted.

11. Use of an animal according to claim 10 as an experimental model for angiogenesis, cancer, macular degeneration, psoriasis, arthritis, multiple sclerosis or atherosclerosis.

12. Use of an animal according to claim 10 for screening for compounds modulating angiogenesis, cancer, macular degeneration, psoriasis, arthritis, multiple sclerosis or atherosclerosis.

13. A method of or for identifying an inhibitor of GPR4 (e.g. human GPR4) for use in a method of manufacturing a medicament for the treatment of a human patient afflicted with a disease selected from; cancer (such as a solid tumour cancer), COPD, arthritis (e.g. rheumatoid arthritis) which method comprises;
(a) contacting a candidate inhibitor with GPR4 (for example human GPR4 expressed on a host cell);
(b) observing an inhibition of GPR4 function (by for example measuring pH dependent cAMP formation);
(c) selecting an inhibitor which demonstrates inhibition of pH dependent cAMP formation;
(d) optionally structurally modifying the inhibitor of step (c) to increase GPR4 function inhibition and/or improve toxicity profile of said inhibitor in the intended human patient.


SEQUENCE LISTING (42 sequences)

SEQ ID NO: 1 - DNA, 21 bp, Artificial
  synthetic small interfering nucleic acid
gcgctgtgtcctatctcaatt

SEQ ID NO: 2 - DNA, 21 bp, Artificial
  synthetic small interfering nucleic acid
ttgagataggacacagcgcag

SEQ ID NO: 3 - DNA, 19 bp, Homo sapiens
gcgctgtgtcctatctcaa

SEQ ID NO: 4 - DNA, 21 bp, Artificial
  synthetic small interfering nucleic acid
ccatgtctggccagataaatt

SEQ ID NO: 5 - DNA, 21 bp, Artificial
  synthetic small interfering nucleic acid
tttatctggccagacatggcg

SEQ ID NO: 6 - DNA, 19 bp, Homo sapiens
ccatgtctggccagataaa

SEQ ID NO: 7 - DNA, 21 bp, Artificial
  synthetic small interfering nucleic acid
cataagaccgcaattctaatt

SEQ ID NO: 8 - DNA, 21 bp, Artificial
  synthetic small interfering nucleic acid
ttagaattgcggtcttatgtt

SEQ ID NO: 9 - DNA, 19 bp, Homo sapiens
cataagaccgcaattctaa

SEQ ID NO: 10 - DNA, 21 bp, Artificial
  synthetic small interfering nucleic acid
ggaggtaggactaacaatatt

SEQ ID NO: 11 - DNA, 21 bp, Artificial
  synthetic small interfering nucleic acid
tattgttagtcctacctccct

SEQ ID NO: 12 - DNA, 19 bp, Mus musculus
ggaggtaggactaacaata

SEQ ID NO: 13 - DNA, 21 bp, Artificial
  synthetic small interfering nucleic acid
gggtctgaagggggaacaatt

SEQ ID NO: 14 - DNA, 21 bp, Artificial
  synthetic small interfering nucleic acid
ttgttcccccttcagaccctg

SEQ ID NO: 15 - DNA, 19 bp, Mus musculus
gggtctgaagggggaacaa

SEQ ID NO: 16 - DNA, 19 bp, Artificial
  synthetic small interfering nucleic acid
ttgagataggacacagcgc

SEQ ID NO: 17 - DNA, 19 bp, Artificial
  synthetic small interfering nucleic acid
tttatctggccagacatgg

SEQ ID NO: 18 - DNA, 19 bp, Artificial
  synthetic small interfering nucleic acid
ttagaattgcggtcttatg

SEQ ID NO: 19 - DNA, 19 bp, Artificial
  synthetic small interfering nucleic acid
tattgttagtcctacctcc

SEQ ID NO: 20 - DNA, 19 bp, Artificial
  synthetic small interfering nucleic acid
ttgttcccccttcagaccc

SEQ ID NO: 21 - DNA, 19 bp, Artificial
  synthetic primer sequence
tgtgctaccgtggcatcct

SEQ ID NO: 22 - DNA, 20 bp, Artificial
  synthetic primer sequence
aaagcacaccagcacaatgg

SEQ ID NO: 23 - DNA, 24 bp, Artificial
  synthetic primer sequence
ttgtcaagctcatttcctggtatg

SEQ ID NO: 24 - DNA, 23 bp, Artificial
  synthetic primer sequence
tggtccagggtttcttactcctt

SEQ ID NO: 25 - DNA, 19 bp, Artificial
  synthetic primer sequence
tgtgctaccgtggcatcct

SEQ ID NO: 26 - DNA, 25 bp, Artificial
  synthetic primer sequence
cttgagttctgacattctccctctt

SEQ ID NO: 27 - DNA, 23 bp, Artificial
  synthetic primer sequence
cagggctgcttttaactctggta

SEQ ID NO: 28 - DNA, 23 bp, Artificial
  synthetic primer sequence
gggtggaatcatattggaacatg

SEQ ID NO: 29 - DNA, 35 bp, Artificial
  synthetic primer sequence
ctggccatactggccggatgtggctcagttgttac

SEQ ID NO: 30 - DNA, 39 bp, Artificial
  synthetic primer sequence
ccgctcgagtcatgcttataccagcggtgtcatgcttat

SEQ ID NO: 31 - DNA, 30 bp, Artificial
  synthetic primer sequence
ccatcgatggctggcagataaggacagacg

SEQ ID NO: 32 - DNA, 36 bp, Artificial
  synthetic primer sequence
ataagaatgcggccgcagcctcttcagtgactatcc

SEQ ID NO: 33 - DNA, 25 bp, Artificial
  synthetic primer sequence
tgatattgctgaagagcttggcggc

SEQ ID NO: 34 - DNA, 22 bp, Artificial
  synthetic primer sequence
cacttcctctccctcctatttg

SEQ ID NO: 35 - DNA, 21 bp, Artificial
  synthetic primer sequence
agcgcatcgccttctatcgcc

SEQ ID NO: 36 - DNA, 19 bp, Artificial
  synthetic primer sequence
ccagcactgtaagaccttc

SEQ ID NO: 37 - DNA, 20 bp, Artificial
  synthetic primer sequence
cgtgcttgttaagcgaatac

SEQ ID NO: 38 - DNA, 20 bp, Artificial
  synthetic primer sequence
agtcattccagaagcctaga

SEQ ID NO: 39 - DNA, 20 bp, Artificial
  synthetic primer sequence
gctgccatgtggactctcga

SEQ ID NO: 40 - DNA, 20 bp, Artificial
  synthetic primer sequence
caggaaggcgatgctgatat

SEQ ID NO: 41 - DNA, 20 bp, Artificial
  synthetic primer sequence
gctcacatgggaatgttcac

SEQ ID NO: 42 - DNA, 22 bp, Artificial
  synthetic primer sequence
atgttgtcaaagttgtcataag